(19) United States
(12) Patent Application Publication
(10) Pub. No.: US 2018/0243126 A1
(43) Pub. Date: Aug. 30, 2018

(54) PORTABLE BLOWER FOR SUPPLYING VENTILATION TO WOUNDS

(71) Applicant: Simple Solutions Inc., Preston, ID (US)

(72) Inventor: Terrel Porter, Preston, ID (US)

(21) Appl. No.: 15/908,697

(22) Filed: Feb. 28, 2018

Related U.S. Application Data
(60) Provisional application No. 62/464,752, filed on Feb. 28, 2017.

(51) Int. Cl. A61F 7/00

(52) U.S. Cl. CPC A61F 7/0085; A61F 2007/006; A61F 2007/0052

(57) ABSTRACT

A portable blower includes a tube that may be adjusted to place air flow at a desired location and in a desired orientation, for example, on a feverish location, a damp location, and/or a wound on an individual's body while the individual convalesces. A method of using such a portable blower includes placing a housing of the portable blower at a location (e.g., outside of bedsheets or blankets, etc.) where the portable blower can take in air, and positioning a distal end of a hose whose proximal end has been coupled to an output of the portable blower at a location and in an orientation where ventilation will be provided to a desired location on an individual's body (e.g., a location covered by a gown, clothing, and/or bedsheets or blankets, etc.). The hose may retain the configuration into which it is adjusted.

### CROSS-REFERENCE TO RELATED APPLICATION

A claim for the benefit of priority to the Feb. 28, 2017 filing date of U.S. Provisional Patent Application No. 62/464,752, titled PORTABLE BLOWER FOR SUPPLYING VENTILATION TO WOUNDS (“the '752 Provisional Application”) is hereby made pursuant to 35 U.S.C. § 119(e). The entire disclosure of the '752 Provisional Application is hereby incorporated herein.

### TECHNICAL FIELD

This disclosure relates generally to devices for ventilating wounds and, more specifically, to portable blowers with tubes that may be shaped to deliver air to desired locations on a subject's body. Methods for ventilating wounds are also disclosed.

### RELATED ART

As an individual convalesces, recovering from surgery, internal injuries, burns, and/or other wounds, the individual's wounds and/or other parts of their body may become heated (e.g., feverish, etc.) as their body heals or as a result of prolonged pressure from lying in bed. The increased temperatures may themselves be uncomfortable for the individual. Further discomfort may be caused by sweating, which is often a body's natural response to increased temperatures, the evaporative effects of which may cool the body. As an individual lies in bed in a hospital or any other environment, wound dressings, a gown or clothing worn by the individual, and sheets and blankets on the bed in which the individual lies prevent the sweat from evaporating, and may spread it to other parts of the individual's body. Sweat, along with pressure and/or friction, may irritate wounds and cause sores, both of which are common among individuals who are confined to bed or otherwise spend considerable amounts of time convalescing in bed, and may lead to more serious problems, such as increased pain and even infection.

Hospitals are often equipped with portable units that provide localized heating and/or cooling to control the temperatures of selected areas of an individual's body, but the availability of these portable heating/cooling units is limited due to the amount of space they consume and due to their cost (they are relatively expensive). Further, they do not circulate air, or provide ventilation. While portable heating/cooling units may be used to reduce the temperature of a selected part of an individual's body, they do not aid in the evaporation of sweat from the individual's body.

### DISCLOSURE

A portable blower according to this disclosure is a relatively small, light-weight, low-cost apparatus that may be easily stored and readily provided to an individual. Such a portable blower may provide the individual with relief from any increase in temperature he or she may experience and from any sweat that may result from the increased temperature. Such a portable blower may direct ventilation to one or more hot spots on the individual's body, which may cool the hot spots and facilitate the evaporation of sweat from those locations and from any dressings, gowns, clothing, sheets, and/or blankets the lie against those locations. By removing heat and/or sweat, the portable blower may reduce the likelihood that the individual will experience increased pain, additional sores, and infection.

In various embodiments, a portable blower according to this disclosure may include a housing, a fan within the housing, and a tube for directing air blown by the fan to a desired location. A filter (e.g., a HEPA (high-efficiency particle arrestance) filter, etc.) may be positioned between the air intake and the fan and/or between the output of the fan and the tube.

The housing of the portable blower may comprise an enclosure for the fan, and carry a power supply (e.g., a cord for supplying alternating current (AC), a battery for supplying direct current (DC) and an accompanying charging cord, etc.) for the fan. The housing includes an air intake (e.g., a vent, etc.) that supplies air from outside of the housing to the fan inside of the housing. A shape and size of the housing may enable it to be positioned on a bed next to an individual as the portable blower supplies air, or ventilation, to a part of the individual's body.

The fan of the portable blower may deliver air at a single volumetric flow (i.e., a single speed), or at a plurality of volumetric flows (i.e., a plurality of speeds). The range of volumetric flows provided by the fan may be a function of an amount of power that is supplied to the fan (a plurality of different amounts of power may be supplied to the fan, e.g., by way of a switch, a dial, or another selector), a configuration of a motor of the fan, and/or a configuration of a blade of the fan.

The tube may comprise an elongated element secured to the housing and positioned over an output of the fan. The tube may communicate air flow generated by the fan to a desired location, such as a location on a body of the individual. In some embodiments, a shape of the tube may be altered to tailor it to the needs of a particular individual. Thus, the tube may be flexible. A flexible tube may be flaccid (e.g., it may comprise a flexible hose, etc.) or it may be rigid (e.g., it may comprise a corrugated, hard plastic, etc.) and adjustable. In some embodiments, a length of the tube may be altered to tailor it to the needs of a particular individual. Such a tube may have a telescoping structure (e.g., a series of concentrically arranged members that may be selectively collapsed and selectively expanded, etc.), it may include corrugations that enable it to be selectively collapsed and selectively expanded, or it may have any other suitable configuration.

In some embodiments, the portable blower may include a Y-piece secured to the housing and positioned over the output of the housing. The Y-piece may enable two tubes to be secured to the housing and, thus, enable ventilation to be supplied to two different locations on an individual's body.

In use, a portable blower may be positioned at a desired location adjacent to an individual's body. The housing or, more specifically, its air intake may remain uncovered (e.g., by sheets, blankets, etc.) so that airflow into the housing (e.g., as the fan draws air into the housing) remains unimpeded. The tube may be positioned in a manner that will deliver air and ventilation to a desired location on the individual's body (e.g., under a gown, onto a wound, etc.). Positioning of the tube may include imparting the tube with a desired shape and/or imparting the tube with a desired length. With the tube in position, power may be supplied to the fan so that the fan may blow air into the tube and the tube may direct the air onto the desired location on the individual's body.

In a specific embodiment, the tube may be positioned between the individual's legs, toward the individual's torso. Such positioning may enable the blower to keep the entire body dry, which may prevent or even eliminate bed sores. When a blower according to this disclosure is used with an individual who is suffering from bed sores, the ventilation provided by the blower may enable the bed sores to heal within as little as two or three days.

In some embodiments, the volumetric flow of the fan may be adjusted to a desired level. The individual whose body is receiving ventilation from the blower may adjust the output of the fan, as desired.

The portable blower may be occasionally repositioned to ventilate other areas of the individual's body, or a plurality of portable blowers may be used to ventilate different locations of an individual's body.

Other aspects, as well as various features and advantages of various aspects of the disclosed subject matter will become apparent from the ensuing description, the accompanying drawings, and the appended claims.

### BRIEF DESCRIPTION OF THE DRAWINGS

In the drawings:

FIG. 1 depicts an embodiment of a portable blower according to this disclosure, showing an adjustable tube assembled with a housing of the portable blower, with the adjustable tube in a compressed configuration;

FIG. 2 provides a frontal perspective view of the portable blower of FIG. 1, with the adjustable tube in an extended and bent configuration;

FIG. 3 provides a rear perspective view of the portable blower of FIG. 1, with the adjustable tube in another extended and bent configuration;

FIG. 4 shows a proximal end of the tube disassembled form the housing of the portable blower of FIG. 1;

FIG. 5 depicts a replacement tube; and

FIG. 6 illustrates an example of use of a portable blower according to this disclosure.

### DETAILED DESCRIPTION

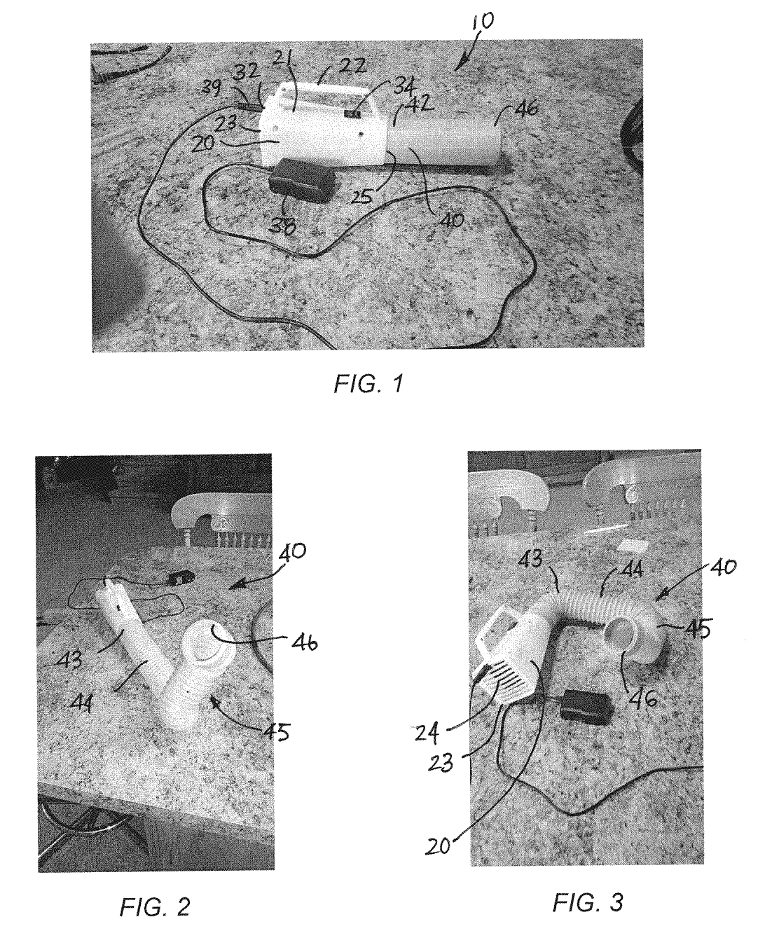

With reference to FIG. 1, an embodiment of a portable blower 10 according to this disclosure is illustrated. As depicted, the portable blower 10 includes a housing 20 and a tube 40. The portable blower 10 also includes a motor, and a fan, and associated electronics for controlling operation of the motor and the fan, all within an interior of the housing 20 and arranged in a manner understood by those of ordinary skill in the art of electric fans. In some embodiments, the housing 20 may also contain a power supply and/or a battery.

On its exterior, the housing 20 may include a handle 22, which may enable an individual to carry, move, and/or position the housing 20 and the remainder of the portable blower 10 at a desired location and in a desired orientation. As illustrated, the handle 22 may be extend from a top surface 21 of the housing 20.

Power may be supplied to the power supply (if any), the battery (if any), and/or the motor through a power port 32. The power port 32 may comprise a female DC connector, which may be capable of receiving a male DC connector 39 of an AC adapter 38, or an AC/DC converter. Alternatively, the power port 32 may simply comprise an opening through which a power cord for connecting the motor directly to an AC (alternating current) power source may extend.

A switch 34 may be accessible from an exterior of the housing 20 to enable an individual to turn the motor on or off. As depicted, the switch 34 may be positioned adjacent to the handle 22 at a location and/or orientation that enable it to be accessed and controlled by the same hand that is used to grasp the handle 22. The switch 34 may comprise a simple two-way rocker switch. Alternatively, the switch 34 may comprise a dial, which may enable an individual to adjust the speed at which the motor operates and, thus, the speed at which the fan spins and the output of the fan, or the volumetric flow generated by the fan.

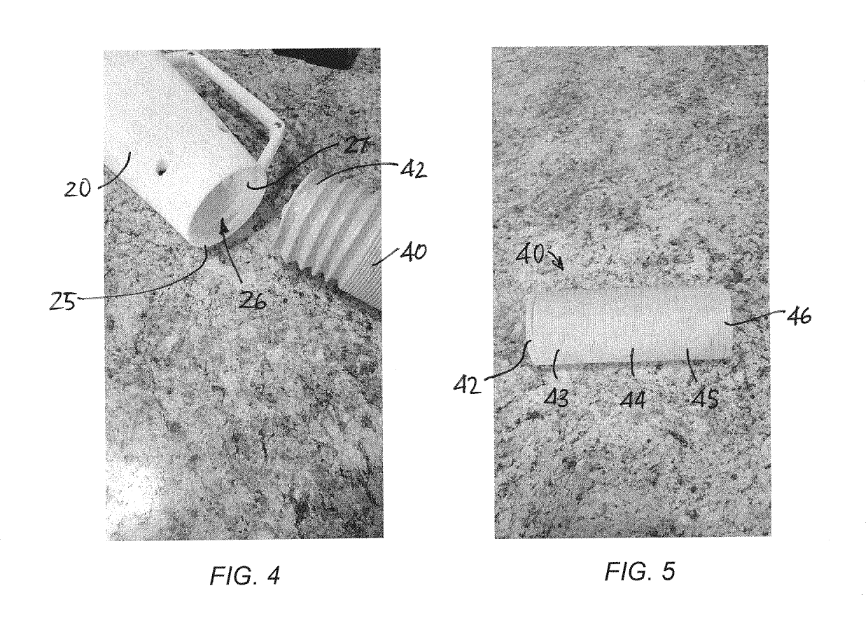

At its rear end 23, the housing 20 includes an air intake vent 24, as illustrated by FIG. 3. As the motor and fan within the housing 20 operate, air is drawn into the interior of the housing 20 through the air intake vent 24. The fan then forces that air out of the interior of the housing 20 through an air output vent 26 in the front end 25 of the housing 20, as depicted by FIG. 4.

FIG. 4 also shows a coupling feature 27 within an interior of the output vent 26 in the housing 20. More specifically, FIG. 4 illustrates a coupling feature 27 that comprises a helical thread on (i.e., protruding from) an interior surface of the output vent 26 of the housing 20. In addition, FIG. 4 illustrates a proximal end 42 of an embodiment of a tube 40 (i.e., the end of the tube 40 to be located closest to the housing 20) that the coupling feature 27 may couple to the housing 20 of the portable blower 10, for example, by “screwing” the proximal end 42 of the tube 40 into the output vent 26 of the housing 20.

FIGS. 1-5 show an embodiment of tube 40 whose length and shape can be adjusted and maintained. More specifically, FIGS. 1-5 shown a corrugated tube 40. It is noteworthy that other embodiments of tubes may also be used in a portable blower 10 according to this disclosure. FIGS. 1 and 5 depict the tube 40 in a compressed state, in which a length of the tube 40 is minimized and the tube 40 has a straight configuration.

FIG. 2 shows the tube 40 in an elongated state, with proximal and central portions 43 and 44, respectively, of the tube 40 having a straight configuration and a distal portion 45 of the tube 40 being bent at two locations to place a distal end 46 of the tube 40 at a desired location relative to the housing 20 and facing in a desired direction (e.g., a location and a direction in which air flow are to be provided, etc.). In FIG. 2, the tube 40 has been formed into a shape that resembles an L.

FIG. 3 shows the tube 40 in another elongated state, with the proximal portion 43 being bent, the central portion 44 being straight, and the distal portion 45 being bent to place the distal end 46 of the tube 40 at another desired location relative to the housing 20 and facing in another desired direction. In FIG. 3, the tube 40 has been placed in a shape that resembles a U.

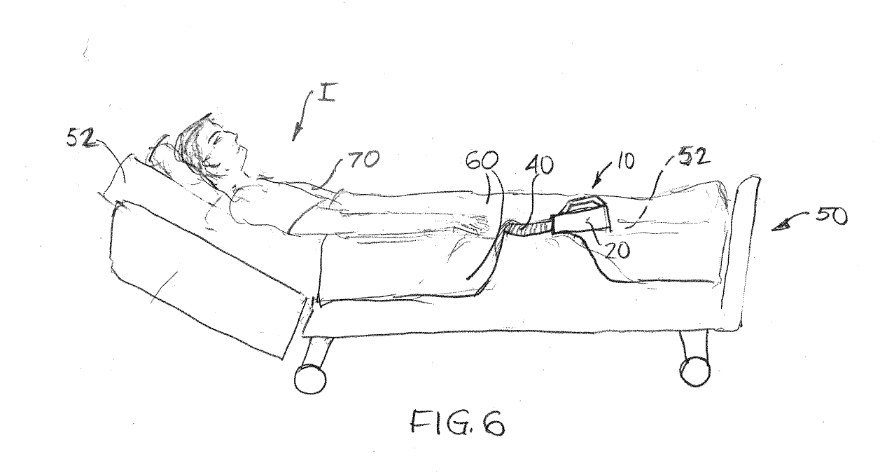

Turning now to FIG. 6, a portable blower 10 may be positioned at a desired location adjacent to an individual I. The housing 20 may be placed on the individual I or close to the individual I on a mattress 52 of a bed 50 on which the individual I is resting. As illustrated, the housing 20 and, more specifically, its air intake vent 24 (FIG. 3) may remain uncovered (e.g., by sheets and/or blankets 60, etc.) so that airflow into the housing 20 (e.g., as the fan draws air into the housing 20) remains unimpeded. The tube 40 of the portable blower 10 may be positioned in a manner that will deliver air and ventilation to a desired location on the individual I's body (e.g., under sheets and/or blankets 60, under a gown 70, onto a feverish location, a damp location, and/or a wound, etc.). Positioning of the tube 40 may include imparting the tube 40 with a desired shape (see, e.g., FIGS. 2 and 3) and/or imparting the tube 40 with a desired length. With the tube 40 in position, power may be supplied to the fan so that the fan may blow air into the tube 40 and the tube 40 may direct the air onto the desired location on the individual I's body.

Of course, the portable blower may be used to direct ventilation onto desired portions of an individual I's body as the individual I sits on a couch, in a chair, in the seat of a vehicle, or at any other time where ventilation of feverish areas, damp areas, or wounds on the individual I's body is desired.

Although the preceding disclosure provides many specifics, these should not be construed as limiting the scope of any of the ensuing claims. Other embodiments may be devised which do not depart from the scopes of the claims. Features from different embodiments may be employed in combination. The scope of each claim is, therefore, indicated and limited only by its plain language and the full scope of available legal equivalents to its elements.

What is claimed:

1. A method for ventilating a desired location on an individuals' body, comprising:
placing a portable blower in proximity to the individual's body;
adjusting a tube of the portable blower in a manner that will direct air onto the desired location of the individual's body; and
supplying power to a fan of the portable blower to deliver air onto the desired location of the individual's body.

2. The method of claim 1, wherein placing the portable blower in proximity to the individual's body comprises placing the portable blower on a bed on which the individual is lying or sitting.

3. The method of claim 1, wherein adjusting the tube includes placing the tube under at least one of a blanket, a sheet, a gown, and clothing over the desired location.

4. The method of claim 1, wherein adjusting the tube comprises shaping the tube.

5. The method of claim 1, wherein adjusting the tube comprises modifying a length of the tube.

6. The method of claim 1, further comprising:
adjusting a volumetric flow output by the fan.

7. A portable blower for ventilating a selected location of an individual's body, comprising:
a housing including an air intake;
a fan contained within the housing and positioned so as to receive air from the air intake of the housing;
a power supply at least partially carried by the housing and capable of supplying power to the fan; and
a tube positioned at an output of the fan and secured to the housing, the tube being capable of being positioned to direct air onto the selected location of the individual's body.

8. The portable blower of claim 7, wherein the tube is capable of being shaped.

9. The portable blower of claim 8, wherein the tube is flexible.

10. The portable blower of claim 9, wherein the tube comprises a rigid material.

11. The portable blower of claim 10, wherein the tube comprises a corrugated tube.

12. The portable blower of claim 7, wherein a length of the tube may be selectively adjusted by compression of the length the tube or expansion of the length of the tube.

13. The portable blower of claim 12, wherein the tube comprises a corrugated tube.

14. The portable blower of claim 7, wherein the power supply is configured to selectively deliver a plurality of different amounts of power to the fan.

15. The portable blower of claim 7, further comprising:
a filter.

16. The portable blower of claim 15, wherein the filter is positioned between the air intake of the housing and the fan.

17. The portable blower of claim 15, wherein the filter is positioned between the output of the fan and the tube.
